(19) United States
(12) Patent Application Publication
(10) Pub. No.: US 2006/0069424 A1
(43) Pub. Date: Mar. 30, 2006

(54) Self-constrained segmented stents and methods for their deployment

(75) Inventors: Pablo Acosta, Newark, CA (US); Craig Welk, Tracy, CA (US); Jeffry J. Grainger, Portola Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Xtent, Inc., Menlo Park, CA (US)

(21) Appl. No.: 10/952,568

(22) Filed: Sep. 27, 2004

(51) Int. Cl. A61F 2/06

(52) U.S. Cl. 623/1.12; 623/1.16

(57) ABSTRACT

A self-expanding stent includes a plurality of segments having a collapsed configuration and an expanded configuration. Preferably, the segments are unconnected to each other in at least the expanded configuration. The segments include restraining structures that temporarily restrain them from expansion until activated. This allows the user to position the desired number of segments at a treatment site and to deploy them simultaneously, thereby avoiding misalignment, overlap, and excessive spacing between segments. In preferred embodiments, multiple segmented stents of user-selectable length may be deployed at multiple locations in a single intervention.

### CROSS-REFERENCES TO RELATED APPLICATIONS

NOT APPLICABLE

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention relates generally to stents for vascular and other applications, and more specifically to self-expanding stents and methods for deploying such stents with greater precision and control.

Stents are tubular prostheses used for scaffolding of arteries and other vessels, fixation of devices such as heart valves and vascular grafts, and other purposes. Stents are generally of two types: balloon expandable or self-expanding. Balloon expandable stents are made of malleable materials and implanted by placing the stent over a tiny balloon at the tip of a catheter, positioning the catheter in a target lumen, and inflating the balloon so that the stent is expanded into contact with the lumen wall. Self-expanding stents are made of resilient or shape memory materials and are deployed by collapsing the stent and retaining it within a tubular catheter, placing the catheter at the target site, and ejecting the stent from the catheter so that it resiliently expands into contact with the lumen wall.

In various applications self-expanding stents have certain advantages. For example, for the treatment of peripheral vascular disease in, e.g., the iliac or femoral arteries, very long and flexible stents are sometimes desirable. Such stents may be deployed over a length of 150 mm or more in tortuous and highly diseased vessels. After deployment, these stents may be subject to very high bending and torsional stresses due to limb movement and patient activity. Thus, highly flexible stents are needed that can be easily deployed over long vascular regions, conform to tortuous vessels, tolerate a high degree of movement and stress, and still provide the necessary vascular scaffolding. For these reasons, self-expanding stents, being more flexible, more easily deployed over long lengths, and capable of providing sufficient radial force to maintain vessel patency, are usually chosen for peripheral vascular applications.

Self-expanding stents do, however, present certain challenges. One such challenge relates to the ability to maintain sufficient control over the stents during deployment to precisely implant them at a desired location. Self-expanding stents have inherent resiliency which allows them to be collapsed down to a small diameter for delivery in a catheter, and which causes them to radially expand when expelled from the catheter. However, this resiliency also can cause such stents to recoil in an uncontrollable fashion when released, wherein the stents jump distally away from the catheter (known as “watermelon seeding”) and/or rotate about their longitudinal or transverse axes. This may result in the stent being placed in a sub-optimal location or orientation relative to the desired treatment site.

Such lack of control can be particularly problematic in applications where more precise stent placement is necessary, such as in the delivery of segmented stents. Segmented stents, such as those disclosed in co-pending application Ser. No. 10/306,813, filed Nov. 27, 2002, the complete disclosure of which is incorporated herein by reference, include a plurality of separate stent segments that must be deployed with controlled inter-segment spacing, without overlap of adjacent segments or excessive space between segments. This requires careful control over the axial position of each segment relative to the adjacent segments. Moreover, interleaving segmented stent designs, such as those disclosed in co-pending application Ser. No. 10/738,666, filed Dec. 16, 2003, the full disclosure of which is incorporated herein by reference, have axially-extending elements on each stent segment that interleave with those on the adjacent stent segment. Such interleaving segments must be deployed so that that not only is optimal axial spacing preserved between segments, but so that adjacent segments maintain the proper rotational position so that the axial elements remain interleaved and do not overlap.

For these and other reasons, self-expanding stents, stent delivery systems and delivery methods are needed which provide greater control during stent deployment for highly precise stent positioning. Such stents, delivery systems and methods should minimize uncontrolled axial and rotational recoil during deployment so that the stents may be deployed accurately and predictably at a desired treatment site. Desirably, such stents, delivery systems and methods will enable the delivery of segmented self-expanding stents in such a way as to maintain optimal inter-segment spacing. Ideally, such stents, delivery systems and methods will provide accurate control over axial motion as well as rotation of segments during deployment so that interleaving segments can be deployed without creating overlap of or excessive spacing between the interleaving elements in adjacent segments.

### BRIEF SUMMARY OF THE INVENTION

The invention provides stents, stent delivery systems, and methods of stent delivery that overcome the challenges outlined above and provide other advantages. The stents, delivery systems and methods of the invention are particularly advantageous for the delivery of self-expanding stents, although the principles of the invention may also be applied to balloon-expandable stents. In preferred embodiments, the invention provides segmented stents, and systems and methods for the delivery of such stents, which enable greater control and precision during stent deployment so that optimal stent position, inter-segment spacing, and relative rotational position of segments is achieved.

In a first aspect of the invention, a stent comprises a plurality of generally tubular self-expanding stent segments axially aligned with each other and being expandable from a collapsed configuration to an expanded configuration, each stent segment being unconnected to the other stent segments in at least the expanded configuration. Each stent segment includes a first strut and a second strut, the first and second struts being closer together in the collapsed configuration than in the expanded configuration. The stent segments further include restraining structure holding the first strut and second struts together to maintain the stent segment in the collapsed configuration, wherein the restraining structure is selectively releasable to allow the stent segment to self-expand into the expanded configuration.

The restraining structure may comprise a head coupled to the first strut and a receptacle coupled to the second strut, the head being releasably engaged by the receptacle. The receptacle may comprise a bump configured to engage the head in the collapsed configuration. Alternatively, the restraining structure may a frangible member extending between the first and second struts. The restraining structures may alternatively comprise structures selected from hooks, loops, barbs, ties, and eyelets. The restraining structure may also comprise a bonding material between the first and second struts, or a coating extending over the first and second struts. The coating may include a bioactive agent, such as one that inhibits hyperplasia. The coating or bonding agent may be durable or biodegradable. The coating, bonding agent or other restraining structure may be adapted to rapidly dissolve when contacted with a fluid. The fluid may be saline or other biocompatible fluid, optionally heated, introduced via a lumen in the catheter. The fluid may also be a body fluid such as blood that contacts selected stent segments by exposing them from a cover or sheath on the catheter. As a further alternative, the coating or bonding agent may be responsive to energy selected from heat, light, ultrasound, magnetic resonance, and X-rays to allow the stent segments to expand. Such energy may be transmitted from a device on the catheter, or may be delivered from a remote source outside the body lumen or outside the patient's body.

Preferably, the stent segments have a combined length of at least about 50 mm, and may have combined length of up to 200 mm or more. In preferred embodiments, each stent segment has interleaving members that axially interleave with interleaving members in an adjacent stent segment in at least the collapsed configuration. The axially interleaving members may also axially interleave in the expanded configuration. The stent segments may be connected to each other in the collapsed configuration or unconnected to each other in both the expanded and collapsed configuration. The stent segments preferably comprise a plurality of closed cells. The closed cells may be bounded at least partially by the first and second struts and the restraining structure may lie within at least one of the closed cells.

The stent segments may be composed of any of various resilient materials suitable for self-expansion. These include superelastic alloys such as nickel-titanium (Nitinol), stainless steels, cobalt chromium, and various polymers. In alternative embodiments, the stent segments may be made of malleable or plastically deformable materials suitable for balloon expansion, such as stainless steel or cobalt chromium. These may be coated with polymers, proteins, therapeutic agents and other materials, both durable and biodegradable, for various therapeutic purposes. In some embodiments for vascular applications, the stent segments are coated with a polymeric carrier containing an anti-hyperproliferative agent such as rapamycin or paclitaxel that gradually elutes from the stent segments into the vessel following implantation.

In a further aspect of the invention, a catheter system for deploying a stent in body lumen comprises a carrier shaft; a plurality of stent segments carried by the carrier shaft, each of the stent segments being self-expandable from a collapsed configuration to an expanded configuration and being axially movable relative to each other in the expanded configuration, each of the stent segments having restraining structure therein maintaining the stent segment in the collapsed configuration; and an activation member that may be selectively actuated to release the restraining structure in one or more stent segments to allow the stent segment to self-expand to the expanded configuration.

The activation member may comprise an expansion member adapted to partially expand the stent segment to release the restraining structure. The expansion member may be an inflatable balloon, a slidable camming head, or other expandable structure. In embodiments in which the expansion member comprises a balloon, the catheter system further includes an inflation lumen fluidly coupled to the balloon.

In some embodiments, a sheath is slidably disposed over the expansion member and retractable to expose a selected portion thereof. The catheter system may further include a pusher adapted to exert a distal force against the stent segments. Preferably, one of the stent segments is positionable outside of the sheath while at least one of the stent segments remains within the sheath. The stent segment outside the sheath remains in the collapsed configuration until the expansion member applies an expansion force thereto. The activation member is preferably adapted to act upon a user-selectable number of stent segments to release the restraining structures in the user-selectable number of stent segments.

In a further aspect of the invention, a method of deploying a stent in body lumen comprises positioning a delivery catheter in the body lumen, the delivery catheter having an activation member and a carrier shaft carrying a plurality of self-expanding stent segments in a collapsed configuration; selecting at least two of the stent segments for deployment, the at least two stent segments being unrestrained from expansion by the catheter and remaining in the collapsed configuration; and actuating the activation member so as to release a restraining structure in the at least two stent segments, wherein upon release of the restraining structure the stent segments self-expand into an expanded configuration in the body lumen.

The body lumen may be any of various anatomical structures, but in preferred embodiments comprises a coronary, femoral, popliteal, tibial, iliac, renal, subclavian, or carotid artery or a vein graft. Other possible target lumens include the biliary ducts, aorta, veins, urethra, trachea, bronchial tubes, esophagus, intestines, fallopian tubes, and heart valves, among others.

Preferably, each stent segment is axially unconnected to other stent segments in the expanded configuration. The stent segments may be completely disconnected in the collapsed configuration, or may be connected in such a way as to disconnect when expanded. In some embodiments, the stent segments axially interleave with one another in the collapsed configuration, and preferably, remain axially interleaved when expanded. The plurality of stent segments may have various lengths. For coronary applications, the stent segments preferably have a combined length of at least about 10 mm, usually about 10-30 mm; for other applications including peripheral vascular treatment, the stent segments have a combined length of at least about 30 mm, often at least about 100 mm, and in some embodiments, at least about 200 mm. Each stent segment may have a length between 2 mm and 100 mm, but in preferred embodiments the segment length is about 4-20 mm.

To enable customizing the length of the deployed prosthesis, the step of selecting the at least two stent segments may comprise selecting a desired number of stent segments to expand based on a target lesion length, and actuating the activation member comprises releasing the restraining structure on the desired number of stent segments. The method may further include retaining at least a third of the stent segments on the carrier shaft while the at least two stent segments expand.

The activation member may operate in various ways to cause expansion of the stent segments. The activation member may partially expand the stent segment to release the restraining structure. In such embodiments, the activation member may comprise an expandable member expandable within the stent segments. Alternatively, the activation member may comprise a camming head slidable through the interior of the stent segments to cause expansion thereof. Various other expanding structures are also possible.

The restraining structure may have various constructions. In an exemplary embodiment, the restraining structure comprises a head coupled to a first strut and a receptacle coupled to a second strut on each stent segment, the head being disposed in the receptacle in the collapsed configuration. The receptacle may have a shape complementary to the head, such as a C-shaped aperture, and may be integrally formed with one or more struts. Alternatively, the receptacle may be a space between two or more struts configured to receive and temporarily retain the head. Heads and receptacles of various shapes, sizes, and configurations are possible. In such cases, releasing the restraining structure comprises removing the head from the receptacle.

In other embodiments, the restraining structure comprises a frangible member extending between first and second struts on each stent segment, and releasing the restraining structure comprises fracturing, tearing, or otherwise separating the frangible member. The restraining structure may alternatively comprise a bonding material between at least a first strut and a second strut on each stent segment, and releasing the restraining structure comprises fracturing, melting, dissolving, or weakening the bonding material. In further embodiments, the restraining structure comprises a coating extending over at least the first and second struts. The coating may be fractured, melted, or otherwise weakened by the activation member in order to allow the stent segments to expand. The coating may also be dissolvable when contacted by a fluid. The fluid may be saline or other biocompatible fluid, optionally heated, introduced via a lumen in the catheter. The fluid may also be a body fluid such as blood that contacts selected stent segments by exposing them from a cover or sheath on the catheter. As a further alternative, the coating may be responsive to energy selected from heat, light, ultrasound, magnetic resonance, and X-rays to allow the stent segments to expand. Such energy may be transmitted from a device on the catheter, or may be delivered from a remote source outside the body lumen or outside the patient's body.

Other aspects of the nature and advantages of the invention will become apparent from the following detailed description when taken in conjunction with the drawings.

### BRIEF DESCRIPTION OF THE DRAWINGS

FIGS. 1A-B are side views of a stent comprising two stent segments according to the invention in collapsed and expanded configurations, respectively.

FIGS. 2A-C are side cross-sectional views of a first embodiment of a delivery catheter according to the invention illustrating the deployment of the stent of FIGS. 1A-B.

FIGS. 2D-E are side cross-sectional views of a second embodiment of a delivery catheter according to the invention illustrating the deployment of the stent of FIGS. 1A-B.

FIG. 3A is a side view of a further embodiment of a stent segment according to the invention in a collapsed configuration.

FIG. 3B is a side view of two of the stent segments of FIG. 3A in an expanded configuration.

FIG. 4A is a side view of another embodiment of a stent segment according to the invention in a collapsed configuration.

FIG. 4B is a side view of two of the stent segments of FIG. 4A in an expanded configuration.

FIGS. 5A-D are side views of a portion of a stent illustrating different embodiments of a restraining structure according to the invention.

FIG. 6A is an oblique view of a further embodiment of a stent according the invention.

FIG. 6B is an end view of the stent of FIG. 6A.

FIGS. 7A-C are side cross-sectional views of another embodiment of a delivery catheter according to the invention illustrating the deployment of the stent of FIGS. 6A-B.

FIG. 8A is a side cross-sectional view of a further embodiment of a delivery catheter illustrating the deployment of another stent according to the invention.

FIG. 8B is a side partial cross-sectional view of still another delivery catheter according to the invention.

FIGS. 8C-D are side cross-sectional views of further embodiments of a delivery catheter illustrating the deployment of another stent according to the invention.

FIGS. 9A-B are side views of another embodiment of a segmented stent according to the invention.

FIGS. 10A-B are side cross-sectional views of a delivery catheter according to the invention schematically illustrating the delivery of a stent like that of FIGS. 9A-B.

### DETAILED DESCRIPTION OF THE INVENTION

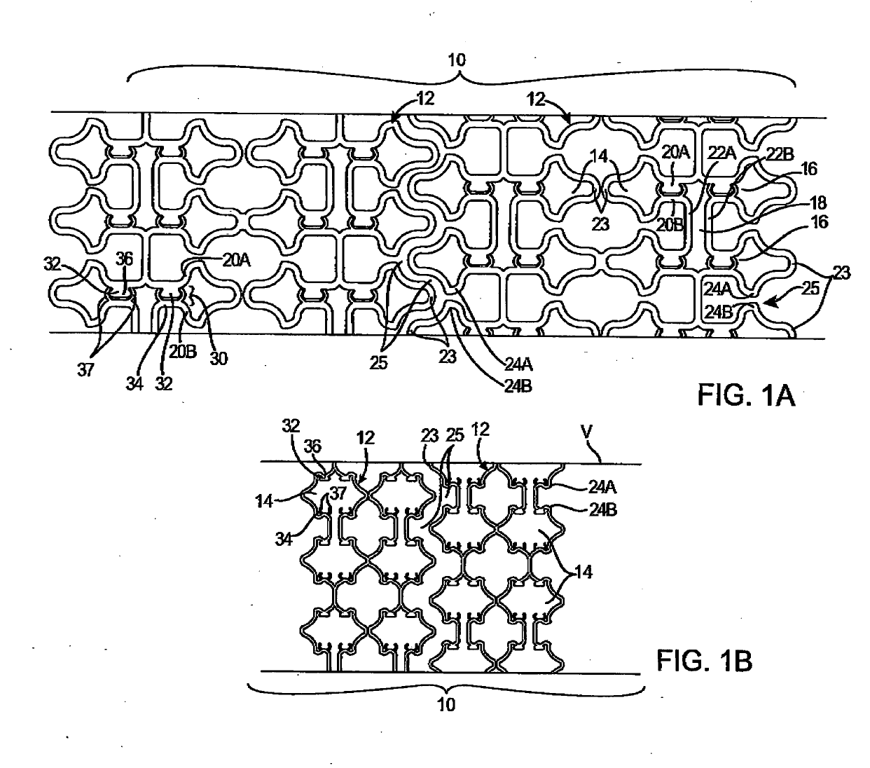

Reference is now made to FIGS. 1A-B, which show a stent 10 according to the invention in a collapsed configuration for delivery (FIG. 1A), and in an expanded configuration in a body lumen V (FIG. 1B). In this embodiment, a stent 10 comprises a plurality of tubular segments 12 that are laser cut from a metal tube into a desired geometry. While a number of preferred stent constructions are described herein, it should be understood that the principles of the invention are applicable to stents of various geometries, materials, and dimensions. Segments 12 may be formed of wire, ribbon, or mesh, cut or etched from a sheet or tube, or molded or woven from polymer, metal, or textile strands, and may be made of various metals, polymers, ceramics, textiles, proteins, or other biocompatible materials. Stent 10 may consist of up to 20 or more segments 12, each being 2-30 mm in length, having a combined length as long as 200 mm or more. In a preferred embodiment, stent 10 is self-expanding, with segments 12 being constructed of a resilient material suitable for being collapsed within a delivery catheter and elastic recoil to an expanded shape when released from the delivery catheter. Suitable materials include nickel titanium alloys such as Nitinol™, cobalt chromium (e.g. MP35N), stainless steels, and elastomeric polymers. It should be understood, however, that the principles of the invention may also be applied to balloon-expandable stents, mechanically expanded stents, hybrid (partially self-expanding, partially balloon expandable) stents, and other tubular prostheses.

Segments 12 may have any geometry suitable to provide the necessary scaffolding of a body lumen when expanded and collapsible into a smaller diameter for delivery with a catheter as described below. In this exemplary embodiment, segments 12 include a plurality of closed cells 14 each comprising a pair of axial slots 16 joined by a circumferential slot 18. Axial slots 16 are bounded on either side by axial struts 20A, 20B, while circumferential slots 18 are bounded by circumferential struts 22A, 22B. Axial struts 20A, 20B are joined at their ends to form rounded tips 23 pointing distally or proximally. Near tips 23 axial struts 20A, 20B have circumferential waves 24A, 24B that form bays 25 between tips 23 adapted to receive tips 23 on the adjacent segment 12, thus providing axial interleaving of adjacent segments 12. In the collapsed configuration, shown in FIG. 1A, waves 24A, 24B may engage the distal or proximal tips 23 of the adjacent segment 12 to maintain suitable axial spacing and relative rotation of segments 12. Except for this engagement, segments 12 are unconnected to each other and free to move axially relative to one another. When expanded, as shown in FIG. 1B, tips 23 remain interleaved, although radial expansion and slight foreshortening of each segment 12 results in increased spacing between adjacent segments 12. Other aspects of stent segments 12 are described in co-pending application Ser. No. 10/738,666, filed Dec.16, 2003, which has been incorporated herein by reference.

In a preferred aspect of the invention, each segment 12 includes a restraining structure 30 that maintains the segment in a collapsed configuration even when unconstrained by an external sheath. In the embodiment of FIGS. 1A-B, restraining structure 30 comprises a tab 32 formed integrally with axial strut 20A and a receptacle 34 formed integrally with axial strut 20B in all or a selected subset of cells 14. Tab 32 is adapted for insertion into receptacle 34 and has a snap-fit or frictional fit therein to provide retention force greater than the self-expansion force of segment 12, thereby maintaining the segment 12 in its collapsed configuration. When an external expansion force is applied to segments 12, e.g. by inflating a balloon within segments 12 as described below, tabs 32 may be urged out of receptacles 34, thereby allowing segments 12 to self-expand into their fully expanded configuration, shown in FIG. 1B. In an exemplary embodiment, tabs 32 have a rounded head-like shape with a narrower neck 36 connecting them to struts 20A. Receptacles 34 have a pair of c-shaped arms 37 forming an opening 38 in which tabs 32 will fit snugly. Arms 37 may be resilient so as to be deflectable apart from each other when an expansion force is applied to segment 12 and resiliently recoiling to their original shape when tabs 32 are released. Alternatively, arms 37 may be constructed to plastically deform when sufficient expansion force is applied to segment 12 to force tab 32 from receptacle 34. Preferably, when segments 12 are radially collapsed, tabs 32 are configured to automatically engage receptacles 34 and be retained therein, thus maintaining segments 12 in the collapsed configuration.

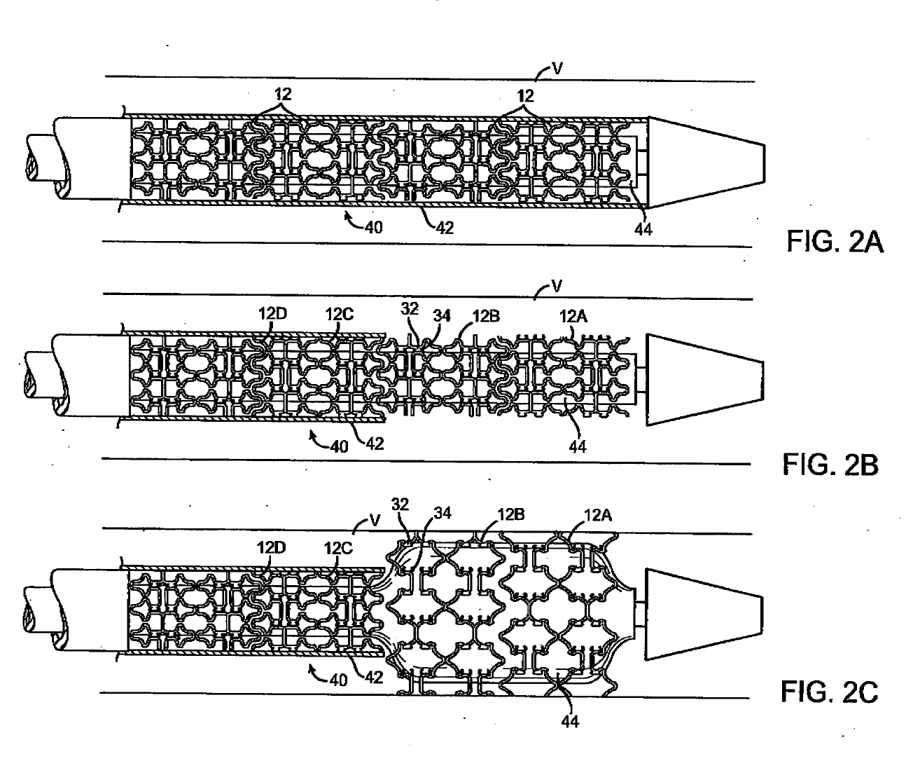

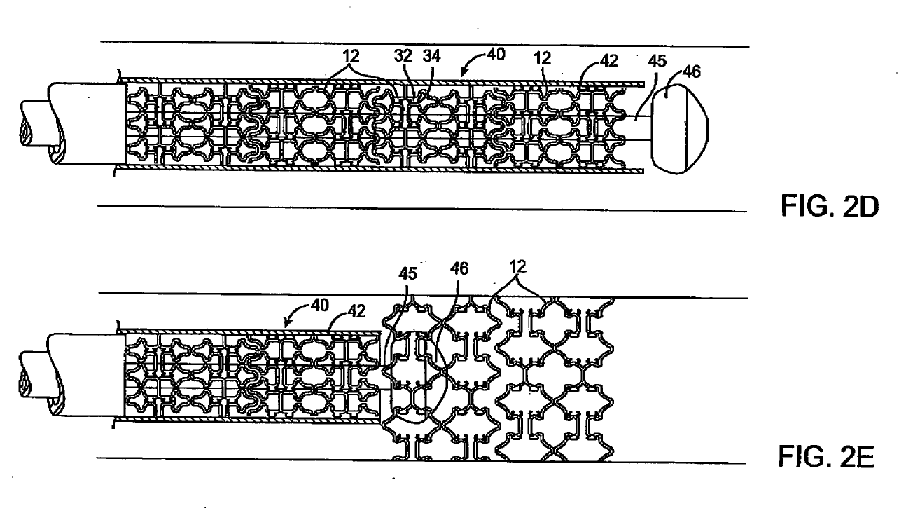

FIGS. 2A-C illustrate the deployment of stent 10 of FIGS. 1A-B. In FIG. 2A a plurality of segments 12 are shown collapsed within a delivery catheter 40. While four segments 12 are illustrated, it will be understood that up to 20 or more segments 12 may be loaded in delivery catheter 40 to enable deployment of one or more stents 10 composed of various numbers of segments 12, without removing catheter 40 from the body between deployments. Once catheter 40 is positioned in the target region of vessel V, sheath 42 on catheter 40 is retracted to expose the desired number of segments 12 corresponding to the length of vessel V to be treated. In the example shown, two segments 12A, 12B are exposed for deployment, while two other segments 12C, 12D are reserved within sheath 42. Although sheath 42 has been withdrawn from around segments 12A, 12B, they remain in a collapsed configuration due to the interconnection of tabs 32 and receptacles 34. When the desired number of segments 12 has been exposed, a balloon 44 is expanded within segments 12 to disengage tabs 32 from receptacles 34. Usually, balloon 44 must expand to a diameter only slightly larger than the collapsed diameter of segments 12 (and somewhat smaller than the diameter of vessel V) in order to release tabs 32. Once released, segments 12 self-expand into engagement with the inner wall of vessel V, as shown in FIG. 2C. Notably, because segments 12 expand simultaneously, axial and rotational alignment and spacing of segments 12 is maintained during expansion, thus maintaining the desired interleaving of segments 12 and preventing excessive space between segments and overlapping of struts. The watermelon seeding and other recoil effects of conventional self-expanding stents are avoided.

Following deployment of segments 12, balloon 44 may be optionally re-expanded into engagement with the interior of segments 12 to post-dilate segments 12, ensuring full expansion thereof and sufficient patency of the vessel V. Balloon 44 may then be deflated, retracted within sheath 42, and catheter 40 repositioned to another location in vessel V for deployment of another stent 10.

FIGS. 2D-E illustrate delivery catheter 40 having an alternative to balloon 44 for applying an expansion force to stent segments 12 so as to disengage tabs 32 from receptacles 34. In this embodiment, in place of balloon 44, an inner shaft 45 extends through segments 12 and is axially movable relative to segments 12 and sheath 42. An enlarged cylindrical camming head 46 is fixed to the distal end of inner shaft 45. Camming head 46 optionally may have a tapered distal end to serve as a nosecone for the delivery catheter, or a separate nosecone may be provided. Camming head 46 is a rigid polymer or metal with a smooth outer surface and a tapered proximal end configured to slide through the interior of segments 12 in contact with the inner surfaces of the struts. Camming head 46 has a diameter slightly larger than the collapsed diameter of segments 12, just large enough to force tabs 32 from receptacles 34 as head 46 is drawn through each segment 12. In use, sheath 42 is first retracted to expose the desired number of stent segments to be deployed, with camming head 46 remaining distal to the exposed segments 12. Inner shaft 45 is then pulled in the proximal direction relative to the exposed segments 12 so that camming head 46 is drawn through the desired number of segments 12 to release. This releases tabs 32 from receptacles 34, thus allowing the exposed segments 12 to expand, as shown in FIG. 2E.

It should be understood that, in addition to balloon 44 and head 46 described above, various types of mechanisms may be used to apply an expansion force to the stents of the invention so as to release the restraining structures therein. These include expandable metal or polymeric baskets, screw-type mechanisms, 4-bar linkages, radially expanding springs, tubular shafts that bulge outwardly when compressed, and other mechanisms capable of providing a radially expansive force to segments 12.

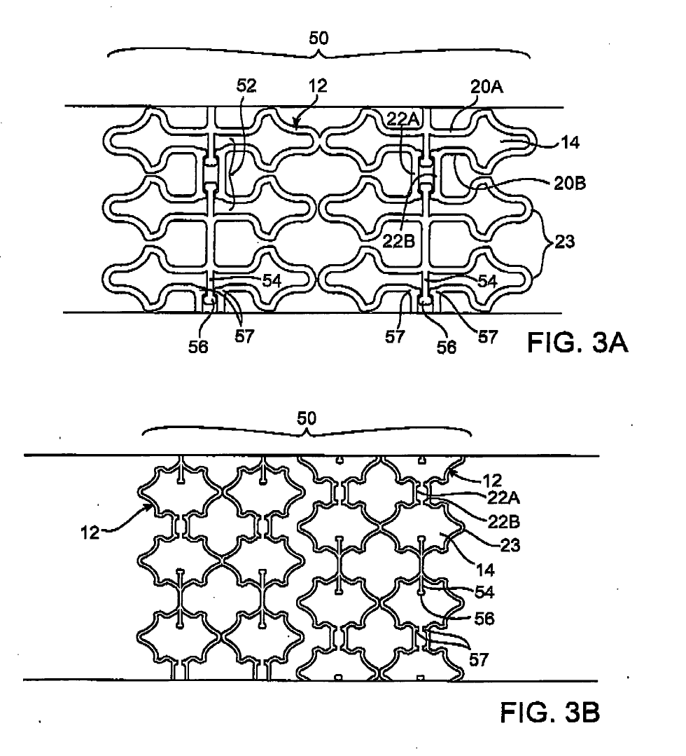

FIGS. 3A-3B illustrate another embodiment of a restraining structure 52 in a stent according to the invention. Stent 50 is constructed similarly to stent 10 described in connection with FIGS. 1A-B, except that in this embodiment, restraining structures 52 comprise extensions 54 that extend from axial struts 20A in the circumferential direction into cells 14 and between circumferential struts 22A, 22B. A pair of opposing bumps 57 are disposed on circumferential struts 22A, 22B, creating a narrowed neck 58 therebetween. Extensions 54 have an enlarged head 56 having a width larger than neck 58 such that heads 56 are trapped between bumps 57 when segments 12 are in the collapsed configuration (FIG. 3A). While the figures show two extensions 54 in each cell 14, in other embodiments the stent may include one extension 54 per cell 14, or may include extensions 54 in only a subset of cells 14. In any event, the force required to extract heads 56 through necks 58 will be greater than the inherent resilient expansion force of the stent so that stent 50 remains in the collapsed configuration until an external expansion force is applied. When sufficient expansion force is applied to segments 12, heads 56 are pulled from between bumps 57, thus allowing segments 12 to self-expand into the expanded configuration shown in FIG. 3B.

To allow heads 56 to pass between bumps 57, circumferential struts 22A, 22B are preferably resilient and flexible enough to deflect away from each other when sufficient force is applied to stent segments 12 (either collapsing or expanding) so that heads 56 push bumps 57 apart, which then recoil back toward each other. Heads 56 and bumps 57 may have various constructions to provide the necessary retention force to maintain segments 12 in the collapsed configuration. For example, heads 56 may be shaped like arrowheads, with tapered points at their distal ends, to facilitate insertion between bumps 57. Bumps 57 may similarly have tapered surfaces on their outer sides to allow easier entry of heads 54. On their proximal sides, heads 54 may be stepped or angular so as to engage the inner sides of bumps 57, which may have a complementary stepped or angular geometry. Alternatively, the proximal surfaces of heads 54 and the corresponding surfaces on bumps 57 may have a reverse taper to facilitate easier withdrawal from neck 58. As a further alternative, heads 56 or the lateral surfaces of extensions 54 may be frictionally engaged by bumps 57 or by circumferential struts 22A, 22B themselves. Further, heads 56 may be barbed or have a Christmas-tree shape so that progressively tighter engagement of heads 56 is achieved by further insertion between bumps 57.

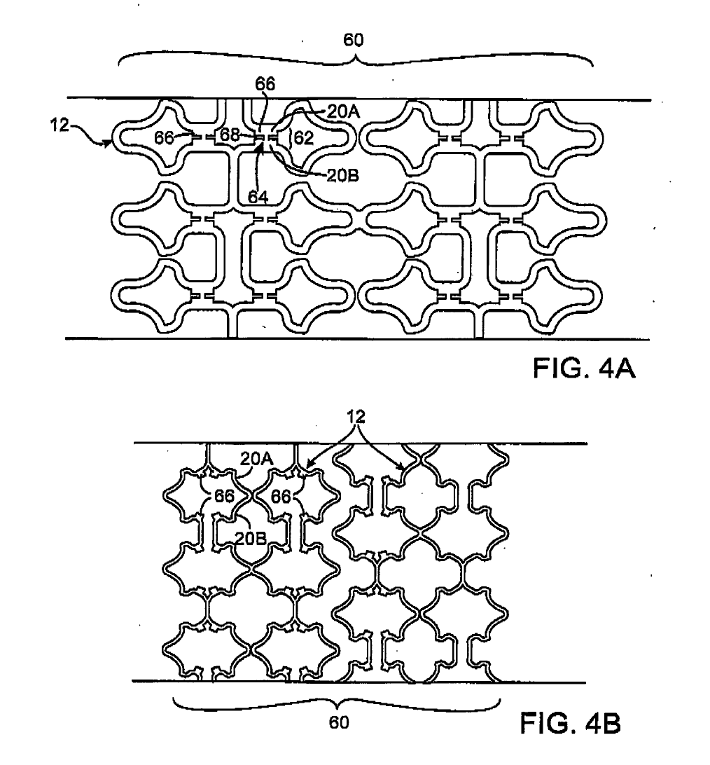

FIGS. 4A and 4B illustrate a stent 60 according to the invention with a further embodiment of a restraining structure 62 therein. In this embodiment, restraining structure 62 comprises a separable member 64 connecting axial strut 20A with axial strut 20B in each of cells 14. Separable member 64 may be formed integrally with struts 20A, 20B, or welded, bonded, soldered, or otherwise attached thereto. Separable members 64 are adapted to separate (sever, tear, or otherwise divide) upon application of sufficient expansion force to segments 12. In one embodiment, separable members 64 each comprise a thin ribbon 66 extending circumferentially between axial struts 20A, 20B and formed integrally therewith. Ribbons 66 have a dent, partial cut, etched line, fold or similar separation region 68 predisposed to separate when tension is applied to ribbon 66. In this way, when expansive force is applied to segments 12, ribbons 66 divide at separation regions 68, allowing segments 12 to self-expand to the configuration of FIG. 4B. In alternative embodiments, separable members 64 may comprise threads, sutures, wires, polymer or textile strands or sheets, or other materials tied, bonded, welded or otherwise attached to axial struts 20A, 20B, and adapted to divide when sufficient force is applied thereto.

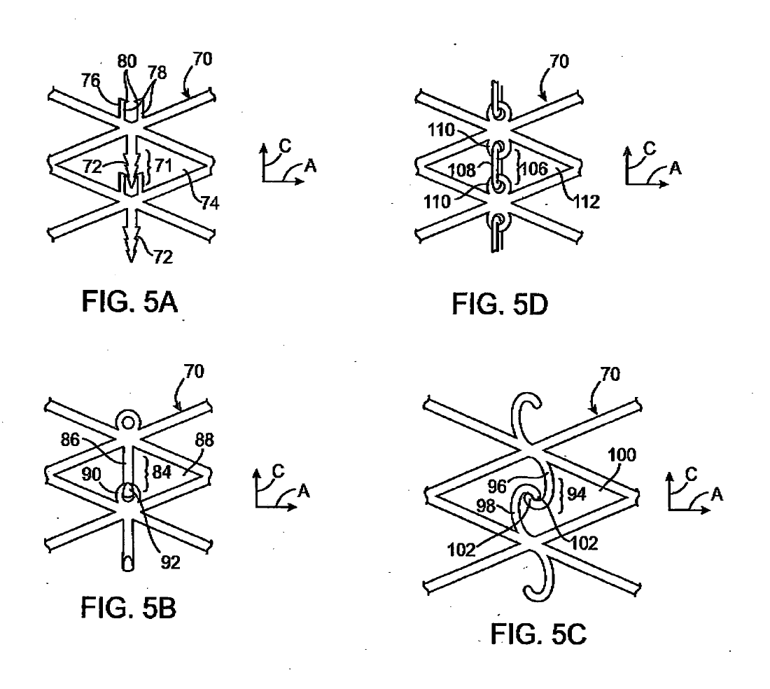

FIGS. 5A-5D illustrate further alternative embodiments of restraining structures according to the invention, wherein axis A indicates the axial direction and axis C indicates the circumferential direction. In these figures, stent 70 is illustrated with diamond-shaped closed cells, but it should be understood that stent 70 alternatively may have the geometry illustrated in FIGS. 1-4, or any other suitable stent geometry. Further, it will be appreciated that the structures illustrated in FIGS. 5A-D may be utilized in single-piece stents or in stents having a plurality of separate segments like those described above.

In FIG. 5A, restraining structure 71 comprises a barbed post 72 extending circumferentially from one side of each cell 74 and engaged by a catch 76 on the opposite side of cell 74. Catch 76 has a pair of opposing arms 78 with inwardly directed tips 80 configured to engage barbed post 72. Arms 78 are resiliently deflected apart as stent 70 is collapsed and barbed post 72 is advanced further into catch 76. Upon application of sufficient expansion force to stent 70, barbed post 72 urges tips 80 outwardly, forcing arms 78 away from each other and allowing barbed post 72 to decouple from catch 76. This permits stent 70 to self-expand into an expanded configuration (not shown), wherein cells 74 widen in the circumferential direction.

FIG. 5B illustrates a further embodiment of a restraining structure 84, comprising a hook 86 extending from one side of cell 88, and a loop 90 on the other side of cell 88. Hook 86 is configured to extend through loop 90 to hold stent 70 in a collapsed configuration. Hook 86 may bend so that its tip 92 is directed either outwardly or inwardly, although in vascular applications it is generally preferred that tip 92 point outwardly so that the interior of stent 70 is smooth to minimize thrombus formation. Hook 86 may be coated with a therapeutic agent such as an anti-hyperproliferative, anti-restenosis, anti-inflammatory, or anti-thrombus agent for elution into the vessel wall or blood stream. Hook 86 may be either resilient or malleable. If resilient, hook 86 is adapted to straighten under sufficient expansion force within stent 70 until it decouples from loop 90 whereupon it springs back to its unbiased hooked shape, allowing cell 88 to widen circumferentially so that stent 70 changes into its expanded configuration. Hook 86 may have a 180° bend so that the surface presented to the vessel wall is smooth, or if desired hook 86 may have a bend of 60°-120° so that its tip 92 engages or penetrates the vessel wall. If malleable, hook 86 straightens as expansive force is applied to stent 70 and, due to plastic deformation, hook 86 remains straight as stent 70 expands, presenting a smooth surface to the vessel wall.

FIG. 5C illustrates a further embodiment of stent 70 having a restraining structure 94 comprising a pair of interlocking hooks 96, 98 extending circumferentially from opposing sides of cell 100. Hooks 96, 98 are bent in the axial direction (around a radial axis) and thus do not protrude either outwardly or inwardly from the stent surface as do the hooks shown in FIG. 5B. When stent 70 is collapsed, hooks 96, 98 are configured to engage each other, deflect axially, and resiliently snap together into interlocking engagement, thus holding stent 70 in its collapsed configuration. When an expansion force is applied to stent 70, hook tips 102 bend until hooks 96, 98 decouple from one another, allowing stent 70 to resiliently expand.

In the embodiment of FIG. 5D, restraining structure 106 comprises a loop 108 extending through a pair of eyelets 110 on opposing sides of cell 112. Loop 108 is configured to break or become decoupled from one or both eyelets 110 upon application of sufficient expansion force to stent 70. Loop 108 may comprise suture, wire, polymeric or textile strands, metal ribbon, or any other suitable biocompatible material. Preferably, loop 108 is fixedly coupled to one or both eyelets 110 so that following breakage, it will remain attached to stent 70. Loop 108 may also be composed of a biodegradable material that gradually is absorbed by the body following stent implantation. Loop 108 could alternatively be adapted to degrade rapidly when exposed to blood or other body fluids so that it would disintegrate when stent 70 was exposed from the delivery sheath within a blood vessel or other body lumen. Stent 70 would then be allowed to expand without need for a balloon or other expansion device to break loop 108. Loop 108 may be a continuous loop or have two free ends which are knotted, twisted, melted, bonded, or interconnected by means of detachable couplings. Loop 108 may alternatively have at least one free end with a T-shaped or other suitable anchoring device designed to insert through one of eyelets 110 and anchor therein to hold stent 70 in a collapsed shape. When sufficient expansion force is applied to stent 70, the anchoring device deforms, breaks, or pulls through eyelet 110 to allow the stent to expand. As a further alternative, a single loop may extend around the circumference of the entire stent 70, threaded in and out of eyelets 110, at one or more axial locations along the stent. As with loops 108 in each cell 112, such circumferential loops would be adapted to break upon application of sufficient expansion force to stent 70, thereby allowing the stent to self-expand.

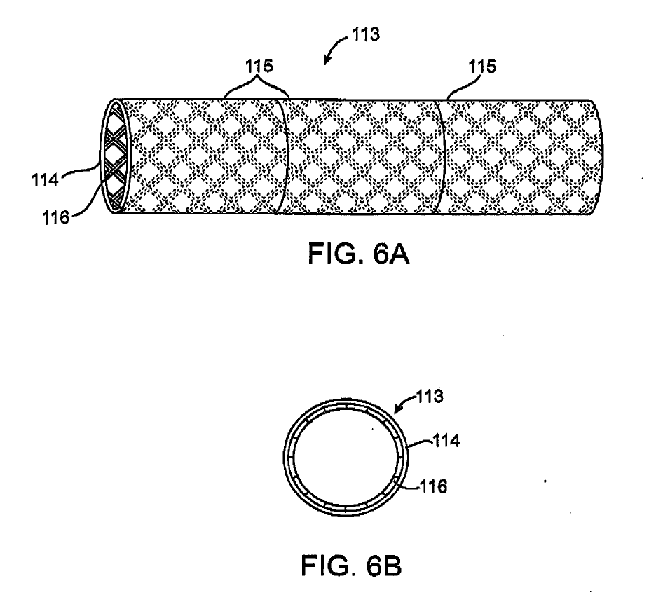

In a further embodiment, shown in FIGS. 6A-B, a segmented stent 113 has a plurality of segments 115 on which a coating 114 is applied to hold stent 113 in a collapsed configuration. Coating 114 is applied on the outer surface of and/or between stent struts 116 and has sufficient strength to hold the stent in its collapsed shape. Coating 114 is adapted to fracture upon application of sufficient expansion force to stent 113 to allow the stent to then self-expand. Suitable coatings may be polymers, sugars, proteins, ceramics, or other materials, and may be impregnated with therapeutic agents such anti-hyperproliferative, anti-restenosis, anti-inflammatory, anti-thrombus and other agents. Alternatively, coating 114 may be applied separately over a coating containing therapeutic agents deposited on stent 113. Preferably, coating 114 is biodegradable or bioabsorbable, but durable coatings may also be used. Coating 114 is preferably brittle or otherwise predisposed to crack, tear or break when an expansion force is applied to stent 70. Coating 114 may also be scored, partially cut, folded, or dented to encourage tearing in particular regions. In segmented stent embodiments, coating 114 may extend continuously over multiple segments 115, or may be discontinuous between segments 115 so that segments 115 are axially movable relative to one another. If coating 114 is continuous across multiple segments 115, it is preferably adapted to break between segments 115 upon segment expansion. To facilitate such breakage, coating 114 may be scored, partially cut, or have reduced thickness around its circumference between segments 115.

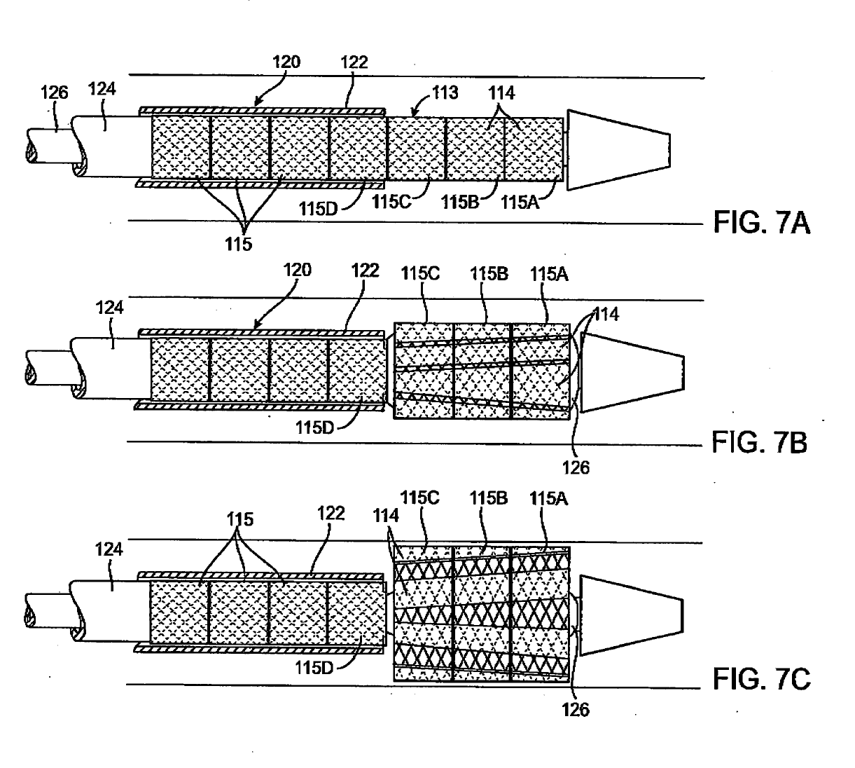

The deployment of stent 113 with coating 114 is illustrated FIGS. 7A-C. Stent 113, comprising multiple segments 115, is carried by a delivery catheter 120 having a sheath 122, a pusher 124, and a balloon 126. Initially, sheath 122 covers all of stent segments 115 during delivery to the treatment site. Once positioned at the target site, sheath 122 is retracted to expose the desired number of stent segments 115 to be deployed, as shown in FIG. 7A. Balloon 126 is then expanded to a diameter large enough to fracture coating 114 over the exposed segments 115A, 115B, 115C. Coating 114 fractures generally axially to allow segments 115A, 115B, 115C to self-expand, as shown in FIGS. 7B-C. Additionally, coating 114 fractures circumferentially between the proximal-most exposed segment 115C and the distal-most unexposed segment 115D within sheath 122. Preferably, coating 114 also fractures circumferentially between each of the exposed segments 115A, 115B, 115C, although in some embodiments this may not be necessary or desirable; coating 114 may be adapted to fracture between segments 115 by natural forces or degradation following deployment in the vessel. Once deployed, as shown in FIG. 7C, coating 114 may elute therapeutic agents into the bloodstream or vessel wall, and preferably gradually biodegrades. Balloon 126 may be retracted back within sheath 122 and the catheter repositioned at another site for deployment of one or more of the remaining stent segments 115.

In addition to fracturable coatings like those just described, other types of coatings, glues, and temporary bonding materials may be used to constrain the stents of the invention in a collapsed configuration. Such materials may be adapted to disintegrate or liquefy when contacted by fluids such as blood, saline, or other chemicals, when heated, or when energized by light, ultrasound, radiofrequency energy, or another energy source. Such materials may be used not only as coatings over all or portions of the stent surface, but may be used to temporarily bond selected stent struts to one another or as temporary bonding agents in restraining structures like those shown in FIGS. 1-5. Such materials may also be used to bond the interior surface of the stent segments to a mandrel or shaft in the delivery catheter to keep the segments collapsed.

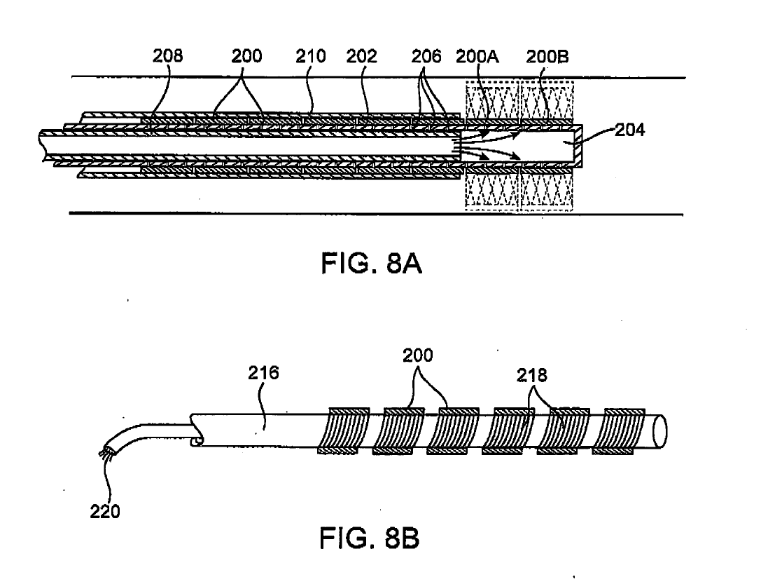

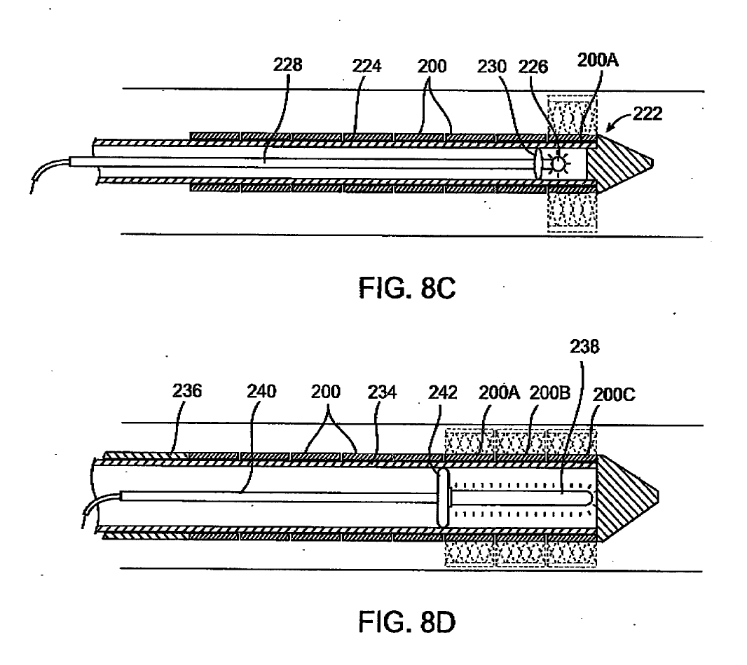

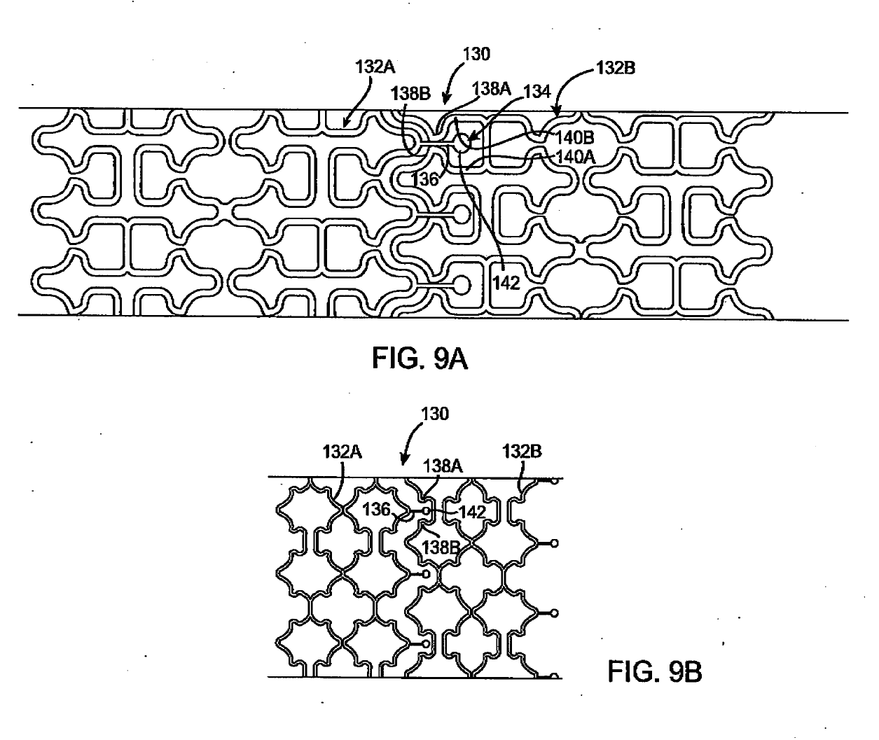

FIGS. 8A-D illustrate alternative delivery devices for delivering stents utilizing such bonding materials. In the embodiment of FIG. 8A, segments 200 are coated or otherwise constrained in a collapsed condition with a bonding agent that dissolves in fluid such as saline. Segments 200 are carried on a tubular carrier shaft 202 having a first lumen 204 and a plurality of sideholes 206 in communication therewith. Stents 200 may be fixed to the exterior of carrier shaft 202 by means of a dissolvable bonding agent, or may be slidable thereon. If slidable, a pusher (not shown) would be slidably disposed over carrier shaft 202 proximal to segments 200 to push segments 200 distally relative to carrier shaft 202. A delivery tube 208 is slidably disposed within carrier shaft 202 and sealingly engages the carrier shaft around its periphery. A sheath 210 is slidably disposed over segments 200 and is in sealing engagement with the exterior thereof. In use, delivery tube 208 and sheath 210 are both retracted relative to segments 200 to expose the desired number of segments to be deployed, with the distal end 212 of delivery tube 208 being just proximal to the proximal-most segment 200A to be deployed. Saline or another suitable fluid, which may optionally be heated, is then delivered through delivery tube 208 into first lumen 204, from which it flows through sideholes 206 and contacts the exposed stents 200A, 200B. This causes the bonding agent on such segments to dissolve, allowing them to self-expand into the vessel. Sheath 210 prevents fluid from reaching the remainder of segments 200 on carrier shaft 202, which thus remain in a collapsed configuration.

FIG. 8B illustrates an alternative embodiment in which the segments 200 are held in a collapsed configuration by a material that melts or weakens when heated. Segments 200 are carried on a shaft 216 and may be either slidable thereon, or bonded thereto by a meltable material. A plurality of heating elements 218, which may be wire coils, heating pads, fluid carrying tubes, or other suitable elements having an axial length approximately equal to that of segments 200, are mounted along the distal portion of shaft 216. Each heating element 218 can be individually activated by means of conductors 220, which extend to the proximal end of the device for connection to a source of electricity, heated fluid, or other appropriate source. One or more control switches (not shown) at the proximal end allow the user to selectively heat one or more of heating elements 218. When one or more heating elements 218 are heated, the segments 200 overlying those heating elements are warmed, causing the constraining material thereon (as well as any material bonding the segments to shaft 216) to weaken or melt. This allows such segments to self-expand into the vessel, while those segments overlying the unheated heating elements 218 remain collapsed on shaft 216.

FIGS. 8C-D illustrate further embodiments in which segments 200 are constrained by means of a material that weakens, melts, or otherwise fails when contacted with light. In FIG. 8C, delivery device 222 includes a tubular carrier shaft 224 made of a material that transmits light, at least at selected wavelengths. Segments 200 are mounted to carrier shaft 224 by means of a light-sensitive bonding agent and thereby maintained in a collapsed configuration. Optionally, an opaque sheath (not shown) may be slidably disposed over segments 200. A light source 226, which may comprise a light emitting diode (LED), optical fiber, incandescent or halogen bulb, or other suitable device which emits light in visible, ultraviolet, infrared or other spectrum, is carried at the end of an inner shaft 228 slidably disposed within carrier shaft 224. A reflector 230 is mounted to inner shaft 228 just proximal to light source 226 and is opaque so as to prevent light transmission proximally thereof. To deploy a selected segment 200A, light source 226 is axially positioned in alignment with segment 200A and illuminated. Light is transmitted through carrier shaft 224 into the bonding agent on segment 200A. The bonding agent weakens and allows segment 200A to self expand into the vessel. Light source 226 may then be repositioned to deploy additional segments 200.

The embodiment of FIG. 8D is similar to that of FIG. 8C, but allows multiple segments 200 may be deployed simultaneously. Here, segments 200 are slidably disposed on a translucent carrier shaft 234 and are maintained in a collapsed configuration by means of a light sensitive material that coats or bonds portions of the segments together. A pusher 236 is slidably mounted over carrier shaft 234 to allow the user to selectively push segments 200 distally relative to carrier shaft 234. An opaque outer tube (not shown) may optionally be slidably disposed over pusher 236 and segments 200. Light source 238 comprises an elongated fiber bundle, LED, bulb, or other suitable light emitter with an axial length as long as at least two segments 200, and preferably as long as the total combined length of segments 200. An opaque sheath 240 is slidably disposed over light source 238 and has an opaque reflector 242 mounted to its distal end. To deploy stents 200, sheath 240 is retracted to expose a length of light source 238 coextensive with the number of segments 200 to be deployed. Light source 238 is then illuminated, thereby weakening the bonding agent in the selected segments 200A, 200B, 200C, which then self-expand into the vessel. Pusher 236 may then be advanced to push the remaining segments 200 to the distal end of the carrier shaft 234, and the device repositioned to deploy additional segments.

In other embodiments, the stents or stent segments of the invention may be coated with materials or utilize constraining structures that are responsive to ultrasound, RF energy, magnetic resonance, X-rays (fluoroscopy) and other forms of energy transmission. In such cases, a delivery device like that shown in FIGS. 8C-D may be utilized, with light sources 226 or 238 replaced with a suitable energy emission device such as an ultrasound transducer or RF electrode. Such devices may be adapted to contact the interior of the carrier shafts 224, 234, or to directly contact segments 200 to transmit energy thereto. Further, remote energy transmission devices disposed outside the lumen being treated, either in a body cavity or outside the patient's body altogether, may be used to transmit energy to the stents of the invention so as to release them from a collapsed configuration. Such devices may include magnetic resonance generators, ultrasound emitters, UV or IR light sources, fluoroscopic devices, and others. These may be adapted to heat the stents and/or constraining materials thereon to melt such materials, or otherwise weaken, fracture, or detach the constraining materials or structures to release the stents from their collapsed configuration.

In addition to circumferentially constraining stents or stent segments so that they may be selectively released for expansion, it may be desirable in some cases to axially constrain or interconnect stent segments to enable greater control during deployment. In a further aspect of the invention, axial restraining structures are provided on each stent segment that couple segments together when collapsed, but which become disconnected when the segments expand. Preferably, when one segment is to be deployed, the restraining structures will keep that segment coupled to the adjacent undeployed segment long enough to allow the deployed segment to engage the vessel wall and become stabilized before it is released. This will prevent “watermelon seeding” and other undesirable displacement during deployment.

In an exemplary embodiment, shown in FIGS. 9A-B, stent 130 comprises a plurality of segments 132, which may be constructed as described above in connection with FIGS. 1-4 and may include any of the restraining structures described above. In this embodiment, segments 132 further include axial restraining structures 134 comprising beams 136 protruding axially from a distal end thereof. Beams 136 are configured to extend between waves 138A, 138B in axial struts 140A, 140B. Beams 136 have enlarged heads 142 which are wider than the gap between waves 138A, 138B when segments 132 are in the collapsed configuration of FIG. 9A, thereby interconnecting segments 132A, 132B. When segments 132A, 132B are in their expanded configuration, shown in FIG. 9B, waves 138A, 138B are further apart, allowing heads 142 to move freely, thereby disconnecting segments 132A, 132B.

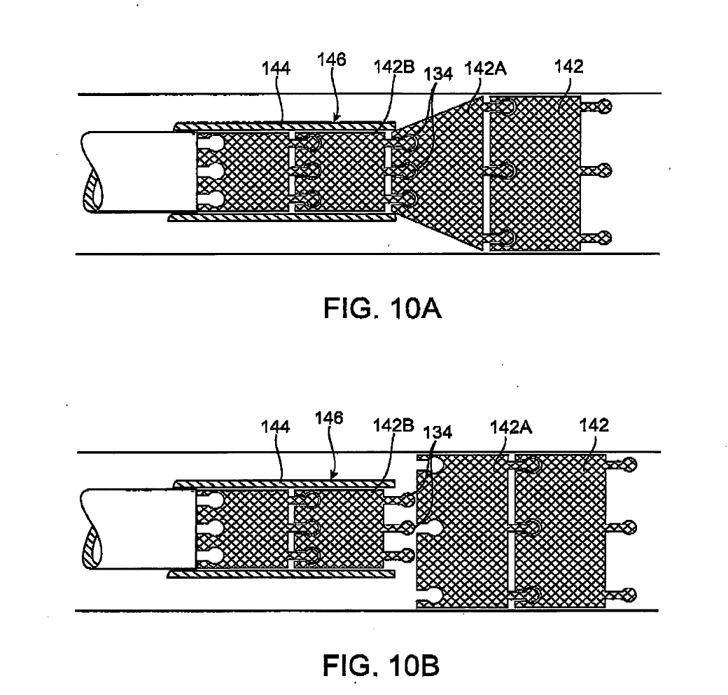

Axial restraining structures 134 are adapted to axially constrain each segment as it is deployed so as to minimize undesirable axial displacement. FIGS. 10A-B schematically illustrate the function of axial restraining structures 134. Sheath 144 on delivery catheter 146 is retracted to sequentially deploy the desired number of stent segments 142 in the vessel. As shown in FIG. 10A, as sheath 144 is gradually retracted, segment 142A progressively expands from its distal end toward its proximal end. As the distal portion of segment 142A expands, restraining structures 134 on the adjacent undeployed segment 142B maintain connection with the proximal end of segment 142A. Preferably, the interconnection of the segments is maintained until the distal end of segment 142A has engaged the vessel wall. This stabilizes the deployed segment 142A and anchors it in position. As sheath 144 is further retracted, the proximal end of segment 142A finally expands and axial restraining structures 134 are released, as shown in FIG. 10B. Because segment 142A is in engagement with the vessel wall, unwanted axial displacement is avoided. This process may be continued for deployment of the desired number of segments.

In addition to the axial restraining structures described above, any of the axial restraining structures described in co-pending application Ser. No. 10/306,813, filed Nov. 27, 2002, or in Ser. No. 10/738,666, filed Dec. 16, 2003, which have been incorporated herein by reference, may also be used in the stents of the invention. It should also be noted that such axial restraining structures may be used in conjunction with the circumferential restraining structures described in connection with FIGS. 1-8. In such embodiments, the stent segments selected for deployment are adapted to expand simultaneously so the need to axially restrain the stent segments to prevent displacement is reduced. However, the use of axial restraining structures helps to maintain axial spacing and rotational alignment of adjacent segments as they expand.

While the above is a complete description of the preferred embodiments of the invention, various alternatives, modifications, additions, and substitutions are possible without departing from the scope thereof, which is defined by the claims.

What is claimed is:

1. A stent comprising:
a plurality of generally tubular self-expanding stent segments axially aligned with each other and being expandable from a collapsed configuration to an expanded configuration, each stent segment being unconnected to the other stent segments in at least the expanded configuration, each of the stent segments comprising:
a first strut and a second strut, the first and second struts being closer together in the collapsed configuration than in the expanded configuration; and
restraining structure holding the first strut and second struts together to maintain the stent segment in the collapsed configuration, wherein the restraining structure is selectively releasable to allow the stent segment to self-expand into the expanded configuration.

2. A stent as in claim 1 wherein the restraining structure comprises a head coupled to the first strut and a receptacle coupled to the second strut, the head being releasably engaged by the receptacle.

3. A stent as in claim 2 wherein the receptacle comprises a bump configured to engage the head in the collapsed configuration.

4. A stent as in claim 1 wherein the restraining structure comprises a frangible member extending between the first and second struts.

5. A stent as in claim 1 wherein the restraining structure comprises a bonding material between the first and second struts.

6. A stent as in claim 1 wherein the restraining structure is a coating extending over the first and second struts.

7. A stent as in claim 6 wherein the coating comprises a bioactive agent.

8. A stent as in claim 7 wherein the bioactive agent inhibits hyperplasia.

9. A stent as in claim 6 wherein the coating is biodegradable.

10. A stent as in claim 1 wherein restraining structure is adapted to dissolve when contacted with a fluid.

11. A stent as in claim 1 wherein the restraining structure is adapted to become disconnected when heated.

12. A stent as in claim 1 wherein the restraining structure is adapted to become disconnected when energy is transmitted thereto.

13. A stent as in claim 1 wherein the stent segments have a combined length of at least about 10 mm.

14. A stent as in claim 1 wherein each stent segment has interleaving members that axially interleave with interleaving members in an adjacent stent segment in at least the collapsed configuration.

15. A stent as in claim 1 wherein the interleaving members axially interleave in the expanded configuration.

16. A stent as in claim 1 wherein the stent segments are unconnected to each other in the collapsed configuration.

17. A stent as in claim 1 wherein the restraining structures comprise structures selected from hooks, loops, barbs, ties, eyelets, and frangible elements.

18. A stent as in claim 1 wherein the stent segments comprise a plurality of closed cells.

19. A stent as in claim 18 wherein the closed cells are bounded at least partially by the first and second struts and wherein the restraining structure lies within at least one of the closed cells.

20. A catheter system for deploying a stent in body lumen comprising:
a carrier shaft;
a plurality of stent segments carried by the carrier shaft, each of the stent segments being self-expandable from a collapsed configuration to an expanded configuration and being axially movable relative to each other in the expanded configuration, each of the stent segments having restraining structure therein maintaining the stent segment in the collapsed configuration; and
an activation member that may be selectively actuated to release the restraining structure in one or more stent segments to allow the stent segment to self-expand to the expanded configuration.

21. A catheter system as in claim 20 wherein the activation member comprises an expansion member adapted to partially expand the stent segment to release the restraining structure.

22. A catheter system as in claim 21 further comprising a sheath slidably disposed over the expansion member and retractable to expose a selected portion thereof.

23. A catheter system as in claim 22 wherein at least one of the stent segments is positionable outside of the sheath while at least one of the stent segments remains within the sheath.

24. A catheter system as in claim 23 wherein the stent segment outside the sheath remains in the collapsed configuration until the expansion member applies an expansion force thereto.

25. A catheter system as in claim 20 wherein the activation member is adapted to act upon a user-selectable number of stent segments to release the restraining structures in the user-selectable number of stent segments.

26. A catheter system as in claim 20 further comprising a pusher adapted to exert a distal force against the stent segments.

27. A catheter system as in claim 21 wherein the expansion member comprises a balloon, the catheter system further comprising an inflation lumen fluidly coupled to the balloon

28. A catheter system as in claim 20 wherein each stent segment comprises first and second struts, the first and second struts being closer together in the collapsed configuration than in the expanded configuration, the restraining structure extending between the first and second struts.

29. A catheter system as in claim 28 wherein the restraining structure comprises a head coupled to the first strut and a receptacle coupled to the second strut, the head being releasably received in the receptacle.

30. A catheter system as in claim 29 wherein the receptacle comprises a bump configured to releasably engage the head in the collapsed configuration.

31. A catheter system as in claim 28 wherein the restraining structure comprises a frangible member extending between the first and second struts.

32. A catheter system as in claim 28 wherein the restraining structure comprises a bonding material between the first and second struts.

33. A catheter system as in claim 28 wherein the restraining structure is a coating extending over the first and second struts.

34. A catheter system as in claim 33 wherein the coating comprises a bioactive agent.

35. A catheter system as in claim 34 wherein the bioactive agent inhibits hyperplasia.

36. A catheter system as in claim 32 wherein the coating is biodegradable.

37. A catheter system as in claim 33 wherein the coating is fracturable to allow the stent segment to expand.

38. A catheter system as in claim 20 comprising at least 3 stent segments.

39. A catheter system as in claim 20 wherein the stent segments have a combined length of at least about 10 mm.

40. A catheter system as in claim 20 wherein the restraining structure is adapted to release in response to energy selected from heat, light, ultrasound, magnetic resonance and x-rays.

41. A catheter system as in claim 20 wherein each stent segment has interleaving members that axially interleave with interleaving members in an adjacent stent segment in at least the collapsed configuration.

42. A catheter system as in claim 20 wherein the interleaving members axially interleave in the expanded configuration.

43. A catheter system as in claim 20 wherein the restraining structures comprise structures selected from hooks, loops, barbs, ties, eyelets, and frangible elements.

44. A stent as in claim 20 wherein the stent segments are connected to each other in the collapsed configuration and unconnected to each other in the expanded configuration.

45. A stent as in claim 20 wherein each of the stent segments comprise a plurality of closed cells.

46. A catheter system as in claim 45 wherein the closed cells are bounded at least partially by first and second struts, the restraining structure extending between the first and second struts.

47. A catheter system as in claim 20 wherein the stent segments comprise a superelastic alloy.

48. A catheter system as in claim 47 wherein the alloy comprises nickel-titanium.

49. A method of deploying a stent in body lumen comprising:
positioning a delivery catheter in the body lumen, the delivery catheter having an activation member and a carrier shaft carrying a plurality of self-expanding stent segments in a collapsed configuration;
selecting at least two of the stent segments for deployment, the at least two stent segments being unrestrained from expansion by the catheter and remaining in the collapsed configuration; and
actuating the activation member so as to release a restraining structure in the at least two stent segments, wherein upon release of the restraining structure the stent segments self-expand into an expanded configuration in the body lumen.

50. A method as in claim 49 wherein each stent segment is unconnected to other stent segments in the expanded configuration.

51. A method as in claim 49 further comprising retaining at least one of the stent segments on the carrier shaft while the at least two stent segments expand.

52. A method as in claim 49 wherein selecting the at least two stent segments comprises selecting a desired number of stent segments to expand based on a target lesion length, and actuating the activation member comprises releasing the restraining structure on the desired number of stent segments.

53. A method as in claim 49 wherein the activation member partially expands the stent segment to release the restraining structure.

54. A method as in claim 53 wherein the activation member comprises an expandable member expandable within the stent segments.

55. A method as in claim 54 wherein the activation member comprises a camming head slidable through the interior of the stent segments.

56. A method as in claim 49 wherein the restraining structure comprises a head coupled to a first strut and a receptacle coupled to a second strut on each stent segment, the head being disposed in the receptacle in the collapsed configuration, and releasing the restraining structure comprises removing the head from the receptacle.

57. A method as in claim 49 wherein the restraining structure comprises a frangible member extending between first and second struts on each stent segment, and releasing the restraining structure comprises fracturing the frangible member.

58. A method as in claim 49 wherein the restraining structure comprises a bonding material between at least a first strut and a second strut on each stent segment, and releasing the restraining structure comprises melting, dissolving, or weakening the bonding material.

59. A method as in claim 58 wherein the restraining structure comprises a coating extending over at least the first and second struts.

60. A method as in claim 59 wherein the coating is fracturable by the activation member.

61. A method as in claim 59 wherein the coating is dissolvable when contacted by a fluid.

62. A method as in claim 59 wherein the coating is responsive to energy selected from heat, light, ultrasound, magnetic resonance, and X-rays to allow the stent segments to expand.

63. A method as in claim 49 wherein the plurality of stent segments have a combined length of at least about 10 mm.

64. A method as in claim 49 wherein the plurality of stent segments have a combined length of at least about 100 mm.

65. A method as in claim 49 wherein the plurality of stent segments are interconnected in the collapsed configuration and become disconnected when expanded.

66. A method as in claim 49 wherein the body lumen is selected from the coronary, femoral, popliteal, tibial, iliac, renal, subclavian, or carotid arteries or vein grafts.

67. A method as in claim 49 wherein the stent segments axially interleave with one another in the collapsed configuration.

68. A method as in claim 54 wherein the stent segments remain axially interleaved in the expanded configuration.
